(19) United States
(12) Patent Application Publication
(10) Pub. No.: US 2014/0031772 A1
(43) Pub. Date: Jan. 30, 2014

(54) SYSTEM AND METHOD FOR COLLECTING STEM CELLS

(71) Applicant: NEXT HEALTHCARE, INC., Germantown, MD (US)

(72) Inventors: Wayne Hardy, Baltimore, MD (US); Vininder Singh, Boyds, MD (US); Danton S. Dungy, Scottsdale, AZ (US)

(73) Assignee: NEXT HEALTHCARE, INC., Germantown, MD (US)

(21) Appl. No.: 13/791,010

(22) Filed: Mar. 8, 2013

Related U.S. Application Data
(60) Provisional application No. 61/677,370, filed on Jul. 30, 2012.

(51) Int. Cl. A61M 1/00

(52) U.S. Cl. CPC A61M 1/0023
USPC 604/319; 604/317

(57) ABSTRACT

A system and method for collecting bone marrow exposed during a surgical procedure is disclosed. The system comprises a cannula; a collection element, such as a syringe; a storage element, such as a blood bag of blood tube; a valve assembly; and connecting tubing. The system is a closed system which significantly reduces the risk of microbial contamination during the collection and/or processing of the bone marrow. The method includes collecting bone marrow using the system during a surgical procedure, such as a hip or knee replacement surgery, and processing the collected bone marrow to obtain stem cells. Once obtained, the stem cells can be cryopreserved in a cell bank for later use.

### CROSS-REFERENCE TO RELATED APPLICATIONS

This application claims priority to U.S. Provisional Application Ser. No. 61/677,370, filed on Jul. 30, 2012, entitled SYSTEM AND METHOD FOR COLLECTING STEM CELLS, the disclosure of which is incorporated by reference herein in its entirety.

### BACKGROUND

Stem cells are capable of being used for treatment of various medical conditions through regenerative medicine and cell therapy. Therapeutic uses of stem cells include, for example, treatment of neurological defects, such as neurological trauma (e.g. spinal cord and brain injuries) as a result of accidents or stroke, or neurological disorders such as Parkinson's disease, multiple sclerosis, Alzheimer's disease, and Huntington's disease. Stem cells, such as pluripotent stem cells and multipotent stromal cells, are capable of differentiating into specialized cells depending on their environment, and therefore have the capability of being used to replace and repair damaged tissues. Stem cells obtained from bone marrow (i.e. marrow stromal cells or mesenchymal stem cells) can be particularly useful in repairing damage to bones and cartilage caused by conditions such as, for example, osteoarthritis. Due to their heterogeneity and plasticity, stem cells obtained from bone marrow are also useful for treating other conditions and tissues, such as pancreatic tissue in treatment of diabetes, skin in treatment of wounds, burns, and other skin related disorders, cardiac muscle in treatment of heart disease or disorders, and other muscle tissues.

New cell therapies are rapidly being developed, and improved sources for stem cells are needed. Currently, stem cells can be obtained from umbilical cord tissue, extraction of adipose tissue, pulp from teeth, peripheral blood, and bone marrow. However, obtaining stem cells from these tissues or peripheral blood is time consuming and can require a surgical procedure and/or multiple treatments to obtain the stem cells. For example, obtaining stem cells from peripheral blood requires multiple apheresis treatments and obtaining the stem cells from bone marrow involves a painful procedure in which a large needle is inserted into the pelvis to aspirate bone marrow cells from the iliac crest.

Another drawback to these conventional stem cell collection procedures is that therapeutically useful amounts of stem cells are not generally obtained and expansion of the stem cells is required to obtain a therapeutically useful amount of stem cells. While significant advancements have been made in the field of controlled expansion of stem cells, expanding a seed sample of stem cells into a therapeutic dosage of pluripotent or multipotent stem cells with the degree of reliability and precision necessary for therapeutic treatment has not yet been obtained. Therefore, there is a need for a method for providing therapeutically useful amounts of stem cells without expansion that is less invasive and painful to the donor, and is less costly and time consuming than currently available methods.

### SUMMARY

A system and method for collecting bone marrow comprising stem cells from bone marrow exposed during a surgical procedure is disclosed. The system comprises a cannula; a collection element, such as a syringe; a storage element, such as a blood bag or blood tube; a valve assembly; and connecting tubing. The system is a closed system which significantly reduces the risk of microbial contamination during the collection and/or processing of the bone marrow. The method includes collecting bone marrow using the system during a surgical procedure, such as a hip or knee replacement surgery, and processing the collected bone marrow to obtain stem cells. Once obtained, the stem cells can be cryopreserved in a cell bank for later use.

### FIGURES AND TABLES

FIGS. 1A-1C are schematic diagrams of an exemplary embodiment of the system and method for collecting marrow stromal cells.

FIGS. 2A and 2B are schematic diagrams of an exemplary embodiment of the system and method for collecting marrow stromal cells.

FIGS. 3A-3D are schematic diagrams of a valve assembly according to an exemplary embodiment.

FIG. 4 is a flow diagram of the method according to an exemplary embodiment.

### DESCRIPTION

For the purpose of this application, the term “stem cells” is intended to encompass cells capable of regenerating tissue by differentiation or by activating or promoting the division and regeneration of other cells and includes pluripotent stem cells and multipotent stem cells and mesenchymal stem cells in bone marrow.

Stem cells have been found within many different types of tissues in the human body and are most commonly associated with blood, bone marrow, adipose tissue and dental pulp. Extraction of stem cells from each of these sources presents its own challenges. For example, peripheral blood is easy to collect, but are large volume of blood must be collected/treated over multiple treatments to obtain a quantity of stem cells sufficient for a seed sample. Bone marrow aspiration is a painful procedure generally performed under general anesthesia and has some possible side effects related to the invasive nature of the procedure. The procedures related to bone marrow aspiration also usually include the transfer of the specimen from a sterile surgical environment to a non-sterile environment in a system open to ambient air, risking exposure and contamination of the collected cells. Methods to extract stem cells from adipose tissue can be very complex and are still under development. Extraction from dental pulp faces similar challenges as extraction from adipose tissue, in addition to the fact that there is very little dental pulp available per tooth so the number of stem cells obtained from dental pulp is very low compared to other tissues.

Embryonic tissues yield high quantities of stem cells because stem cells are very concentrated in such tissues. However, using embryos to produce stem cells for research and therapeutic purposes is highly controversial. Stem cells from other tissues are not as concentrated due to dilution by other types of cells and are therefore available in much lower concentrations and quantities, typically only producing enough cells to be used as seed cells.

To arrive at therapeutically useful quantities, stem cells obtained by conventional procedures must first be isolated or enriched and then propagated and expanded. Isolation or enrichment of stem cells typically involves the elimination of unwanted differentiated cells, particularly for cells obtained from non-marrow tissues or fluid. This isolation or enrichment can be performed, for example, by targeting markers on the unwanted cells with antibodies, by destroying the cells using complement proteins, or by removing factors that support the survival of the unwanted differentiated cells to reduce the number of those cells. Hematopoietic stem cells, such as stem cells obtained from blood or bone marrow, can also be coarsely separated by gradient centrifugation and further enriched by cell sorting using antibodies that bind to particular identifying surface proteins of the cells.

As conventional collection procures do not generally provide a therapeutically useful amount of stem cells, the isolated or enriched cells must also be expanded to obtain a therapeutically useful amount. While significant advancements have been made in the area of controlled expansion of stem cells, expanding a seed sample of stem cells into a therapeutic dosage of pluripotent or multipotent stem cells with the degree of reliability necessary for therapeutic treatment has not yet been obtained.

The present application provides for a convenient and low cost system and method that allow for the collection of a therapeutically useful amount of stem cells from a patient who is already undergoing another surgical procedure, thus sparing the patient from further pain or discomfort and the risks associated with another procedure, saving time and money by combining two or more procedures into a single procedure, and eliminating the need to expand the harvest cells to obtain a therapeutically useful amount. The system is a closed system in which, once the bone marrow has been collected from the patient, its exposure to the environment and possible microbial contaminants are substantially eliminated.

The marrow of a bone is exposed during certain surgical procedures, such as joint replacement procedures. For example, in hip or knee replacement surgery, osteotomy may be performed on the proximal end of the femur (hip replacement) or the distal end of the femur and proximal end of the tibia (knee replacement), exposing the bone marrow of these bones. In some hip replacement procedures the acetabulum (hip socket) may be prepared in a way that allows for convenient collection of bone marrow from the site. Bone marrow may also be exposed due to trauma to a bone and may be collected during subsequent treatment, providing the patient with stem cells to be used in a cell therapy related to the trauma. According to an embodiment, bone marrow or marrow stromal cells comprising stem cells can be collected either during or after the surgery (or in cases where a section of the bone is removed, collection can be done after the surgery), thus avoiding a separate procedure to collect stem cells from the patient. This provides for a fast and convenient method to extract a therapeutically useful amount of stem cells highly capable of differentiation and proliferation.

Bone marrow or marrow stromal cells collected from the femur or pelvis during hip replacement surgery or the femur and tibia during knee replacement surgery is estimated to contain approximately 0.1-1 million stem cells per milliliter, but the number may vary among patients depending on sex, age, health, and nutrition. The differentiation and proliferation capabilities of stem cells have been found to be largely independent of the age, gender and condition of the donor, suggesting that even elderly patients with arthritic conditions would be viable candidates for harvesting marrow stromal cells from bone marrow during surgery.

Generally between about 20 ml to about 100 ml of bone marrow is collected from the patient during the surgical procedure, which typically provides from about 4,000,000 to about 20,000,000 stem cells. A therapeutic dosage suitable for a microtransplant of cells generally includes at least 4,000,000 stem cells, preferably at least 10,000,000 cells, which can be obtained by the system and method of the disclosure without requiring further expansion of the cells. Specimens collected using the system and method of the disclosure may be processed using conventional bone marrow/stem cell processing methods and used immediately in cell therapy or cryopreserved for later us. For example, the specimen may be filtered, centrifuged and washed to separate the cells from any other material contained in the specimen and resuspended in a medium suitable for cell therapy. Alternatively, the cells are resuspended in a cryopreservation medium, such as CryoStor® (available from BioLife Solutions, Inc. in Bothell, Wash.), and cryogenically preserved in a cell bank for later use using conventional cryogenic freezing mediums and methods.

As the disclosed system and method utilizes a closed system, a safe and hygienic means for collecting and transferring bone marrow from a patient into a storage system is provided that reduces exposure of the collected stem cells to microbial contamination. Cells collected using the system and method of the disclosure can either be collected for donating to another patient, or for the donor's own use, and can be cryogenically stored in a cell bank for later use.

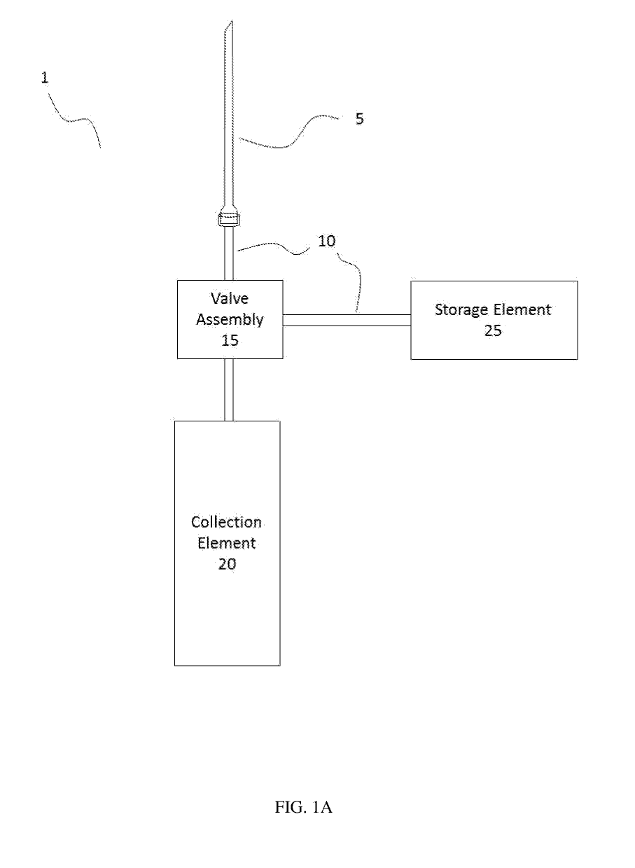

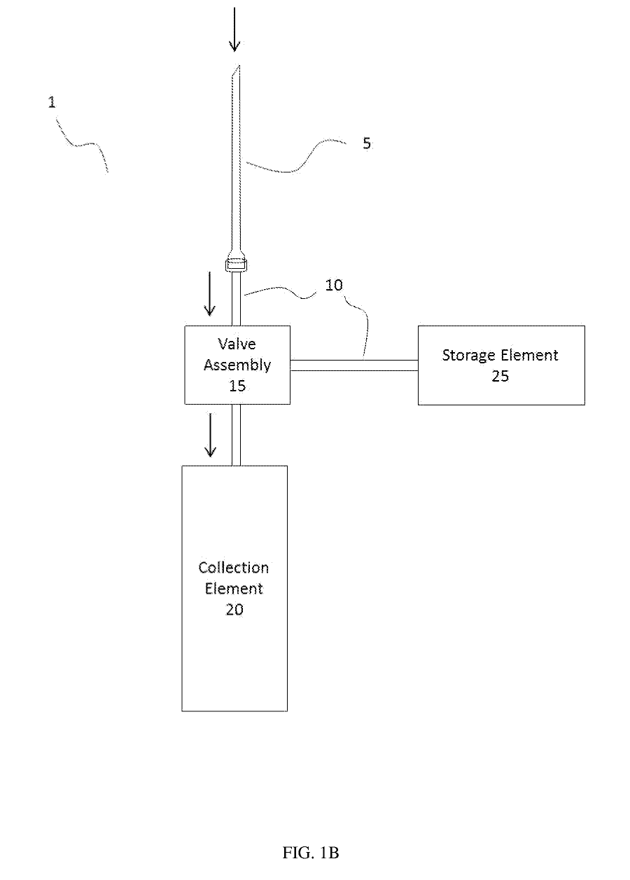

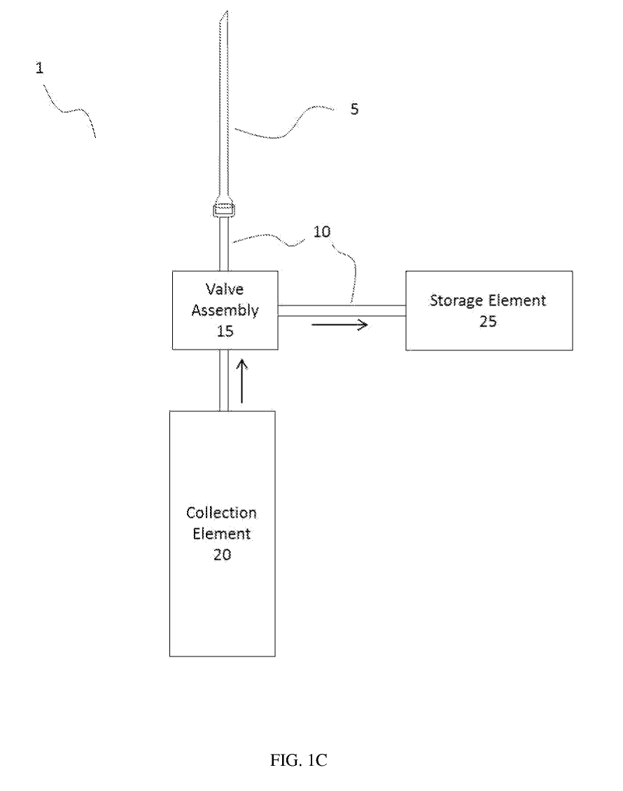

As shown in FIG. 1A, the system 1 generally includes a needle 5, such as a cannula, for collecting the bone marrow during the surgical procedure connected by tubing 10 and a valve assembly 15 to a collection element 20 and a storage element 25. The valve assembly comprises a selectable flow path comprising a first flow path providing fluid communication between the cannula and the collection element and a second flow path providing fluid communication between the collection element and the storage element. The valve assembly can be selectively actuated to provide the desired flow path between the cannula and collection element or the collection element and the storage element. In an embodiment, the valve assembly contains a separate valve to control the flow of bone marrow between the cannula and the collection assembly and the collection element and the storage element. The valve assembly can be manually actuated to direct the flow path through the valve assembly or automatically actuated by positive or negative pressure to control the flow path through the valve assembly. Examples of a collection element suitable for use include, but are not limited, to a vacuum pump or syringe. Examples of a storage element suitable for use include, but are not limited to, blood collection tubes or bags.

According to an embodiment, as shown in FIG. 1B, when bone marrow or marrow stromal cells comprising stem cells is collected from the interior of bone exposed during the surgery procedure, the cannula 5 is inserted into the marrow and suction is applied via the collection element 20 to remove the marrow from the bone. The valve assembly 15 is opened from the cannula 5 to the collection element 20, providing a flow path between the cannula and the collection element allowing the withdrawn marrow to enter the collection element through the valve assembly. After a desired amount of bone marrow has been collected, the marrow is transferred from the collection element 20 to a storage element 25, as shown in FIG. 1C, by opening the valve assembly 15 to provide a flow path between the collection element to the storage element and applying a pressure across the system to transfer the bone marrow into the storage element from the collection element. According to a preferred embodiment, an anticoagulant, such as sodium heparin, is drawn through the cannula into the collection element prior to collecting the bone marrow to reduce or prevent coagulation of the blood cells in the marrow during the collection procedure.

As the system is a closed system, exposure to ambient air is limited during the collection procedure with significantly reduces and/or eliminates the risk of microbial contamination from bacteria, fungi, viruses and the like during collection and processing of the cells. If collection of the specimen from the patient is done in a sterile, surgical environment, the use of the system and method enables the maintaining of the sterile environment, further protecting the specimen from microbial contamination. The transfer of the specimen (e.g. bone marrow) from the collection element to the storage element according to the system and method is also done completely within the closed system thereby eliminating exposure of the cells to ambient air during the transfer process.

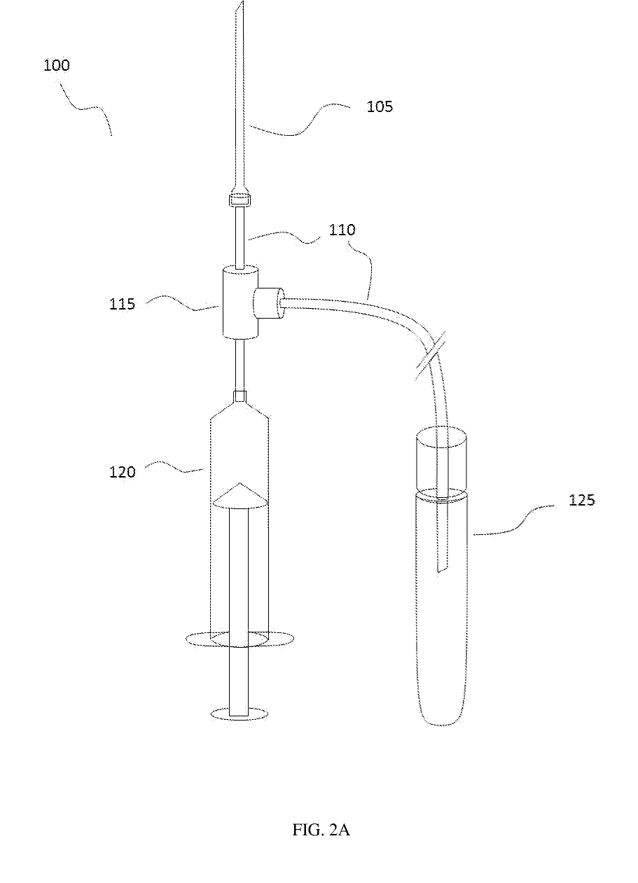

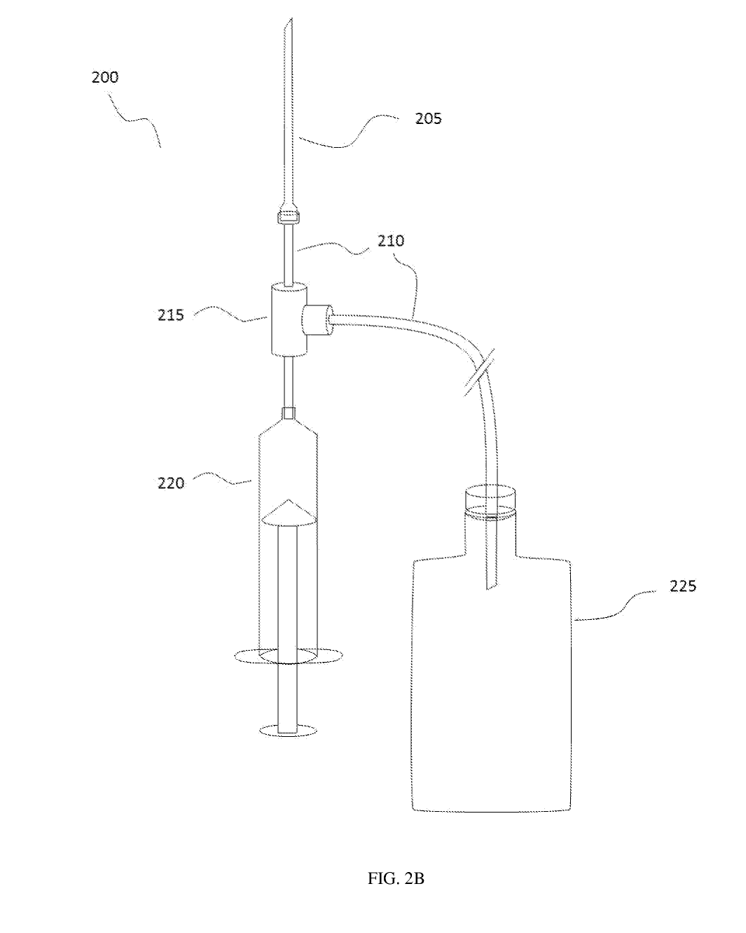

Referring now to FIG. 2A, an exemplary embodiment of the system 100 is shown. The collection element 120 can comprise a syringe, such as, for example a 30 mL or 60 mL collection syringe comprising a plunger. A commercially available syringe, such as a BD Luer-Lok™ Tip syringe (e.g. part no. 309653, available from Becton, Dickinson and Company in Franklin Lakes, N.J.), can be used. The storage element 125 can comprise a bone marrow or blood collection tube, as shown. Commercially available tubes, such as Vacutainer® (available from Becton, Dickinson and Company in Franklin Lakes, N.J.) blood collection tubes, can be used. According to a preferred embodiment, a collection tube containing sodium citrate or ethylenediaminetetraacetic acid (EDTA) can be used to ensure preservation of the collected cells. The cannula 105, valve assembly 115 and the collection 120 and storage 125 elements can be connected to the tubing 110 by detachable connectors, such as, for example, Luer-locks (e.g. Luer-Lok™ available from Becton, Dickinson and Company in Franklin Lakes, N.J.). Commercially available tubing may be used, such as a Fluid Transfer Set (part no. MCA108L available from B. Brown Medical, Inc. in Bethlehem, Pa.).

Referring now to FIG. 2B, an exemplary embodiment of the system 200 is shown. The collection element 220 can comprise a bone marrow collection bag, such as, for example a 150 or 300 mL collection bag. Examples of commercially available bone marrow or plasma collection bags include Teruflex® (available from Terumo Medical Corporation in Somerset, N.J.) and Transfer Pack (e.g. part no. 4R2001, available from Fenwal, Inc. in Lake Zurich, Ill.). The cannula 205, valve assembly 215 and the collection 220 and storage 225 elements can be connected to the tubing 210 by detachable connectors.

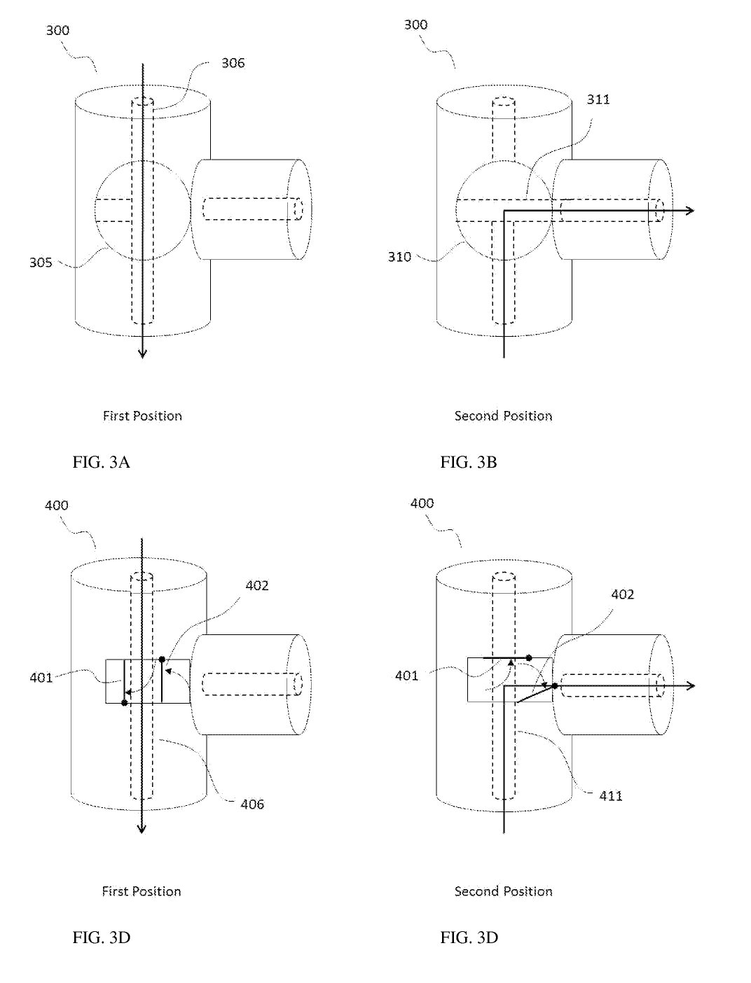

As shown in FIGS. 3A and 3B, the valve assembly 15 comprises a manually operated three-way-valve assembly 300 to direct the flow of bone marrow in the collection system. In the open position 305 shown in FIG. 3A, a flow path 306 between the cannula and collection element is established allowing the bone marrow to flow from the cannula to the collection element. In the closed position 310 shown in FIG. 3B, a flow path 311 between the collection element and storage bone marrow is established allowing the transfer of the bone marrow from the collection element to the storage element. A commercially available valve, such as Baxter part no. 2C6240 (available from Baxter International, Inc. in Deerfield, Ill.) and other 3-way type valves suitable for use in a surgical procedure can be used.

In another embodiment, as shown in FIGS. 3C and 3D, the valve 15 comprises an automatic valve assembly 400. The automatic valve assembly comprises a first 401 and second 402 pressure valve which control the direction of flow through the valve assembly In the automatic valve assembly 400, the opening and closing of the first and second valves is actuated by positive or negative pressure applied from the collection element such that the first valve is opened when the second valve is closed and the first valve is closed when the second valve is opened. For example, when suction is being applied from the collection element, the first valve 401 is opened and the second valve 402 is closed establishing a flow path 406 between the cannula and collection element allowing the bone marrow to flow through the cannula and valve assembly into the collection element and preventing flow of the collected bone marrow to the storage element. When pressure is applied from the collection element, the first valve 401 is closed and the second valve 402 is opened providing a flow path 411 between the collection element and the storage element allowing for the transfer of the collected bone marrow to the storage element.

A method of collecting bone marrow from a patient during a surgical procedure with the system of the disclosure is another aspect of the invention. The method generally includes actuating the valve assembly to provide a flow path between the cannula and the collection element; suctioning the bone marrow into the collection element; actuating the valve assembly to provide a flow path between the collection element and the storage element; and transferring the collected marrow from the collection element to the storage element. The valve assembly can be actuated before or after insertion of the cannula into the bone marrow to provide the flow path between the cannula and the collection element.

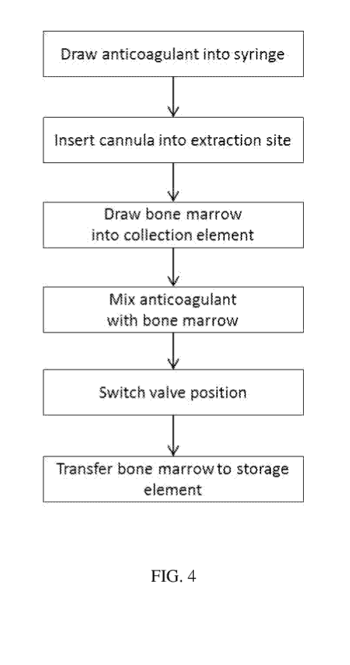

According to an exemplary embodiment, steps performed to collect bone marrow using the system and method are shown in FIG. 4. First, anticoagulant may be drawn into the syringe to prevent coagulation. Alternatively, the system may already comprise an anticoagulant. Next, the cannula is inserted into the extraction site (e.g. bone marrow). The site may be either a site of exposed bone marrow in a patient or a section of bone removed from the patient. For example, during hip replacement surgery the marrow may be collected directly from the acetabulum or iliac crest of the pelvis, the femoral canal, an osteomized section of the femur, other bone fragments obtained during the procedure, or a combination thereof. During knee replacement surgery marrow may be collected from the femoral and/or tibial canal, an osteomized section of femur of tibia bone fragments obtained during the procedure, or a combination thereof. Suction can be applied by the collection element, such as syringe or vacuum pump, initiating the flow of bone marrow into the collection element.

After a desired amount of bone marrow is collected in the collection element, such as a syringe, the syringe can be inverted to mix the collected marrow with the anticoagulant. The valve position is then actuated (either manually or in the case of an automatic valve assembly, by applying a positive or negative pressure) to provide a flow path between collection element and the storage element allowing the collected marrow to be transferred from the collection element into the storage element, such as a tube or a bag for storage and/or processing of the cells.

Depending on the size of the collection element relative to the size of the storage element (e.g. a bag), the syringe may be filled several times and the contents transferred into the storage element. Once the collected marrow has been transferred into the storage element, the storage element can be sealed to prevent leakage and contamination, and may thereafter be disconnected from the rest of the system. The collected specimen contained in the storage element can then be centrifuged as required prior to cooling and/or further processing.

According to an exemplary procedure, a bone marrow or marrow stromal cells is collected in a collection tube, such as a Vacutainer® CPT™ tube (part no. 362761, available from Becton, Dickinson and Company in Franklin Lakes, N.J.) or blood collection bag that contains an anticoagulant (e.g. sodium citrate or EDTA). The tube or bag can be inverted several times to mix the collected cells with the anticoagulant and centrifuged to separate the cells into component cell layers from which the stem cells can be recovered from the buffy coat layer. For example, the specimen may be centrifuged for 10-20 minutes at about 1,000-1,500 rpm and at 18-20° C. The stem cells remain in the buffy coat layer, the middle layer between the top layer comprising mostly plasma and the bottom layer comprising mostly red blood cells, which can be separated, for example, by pipetting. The buffy coat can be concentrated by centrifuging the collected layer for about 10-12 minutes at about 2,000-2,100 rpm and discarding the supernatant. The cells can then be suspended in Dulbecco's Phosphate Buffered Saline (DPBS) and centrifuged for about 10-12 minutes at about 1,000-1,200 rpm. The cells can be resuspended in another volume of DPBS and counted using, for example, a Countess® Automated Cell Counter (part no. C10227, available from Life Technologies Corp. in Grand Island, N.Y.).

Once isolated from the collected bone marrow, the stem cells can be sorted, if desired, using conventional cell sorting techniques, such as flow cytometry, and fluorescently labeled antibodies that bind a particular marker on the surface to identify particle subsets of stem cells from the pool of collected cells. CD31, CD34, CD44, CD45, CD90, CD 105, CD117, and CD166 are several examples of cell surface markers, the presence or absence of which can be used to identify stem cells from other blood cells and the differentiation potential of the stem cells such that the stem cells can be sorted into groups, if desired, of cells based on differentiation potential (e.g., totipotent, pluripotent, multipotent, oligopotent, and unipotent). A listing of useful CD markers for identifying and characterizing stem cells can be found, for example, at www<.>abcam<.>com/CDmarkers.

If the stem cells are to be frozen and banked for later use, the cells are resuspended in a cryogenic storage medium, such as for example, CryoStor™ (part no. CS10, available from StemCell Technologies, Inc. in Vancouver, BC, Canada), to a final concentration of about 4.0×10⁶-10.0×10⁶ cells per milliliter of medium. The suspended cells can be stored in cryogenic storage vials (e.g. CryoELITE™ part no. W985861, available from Wheaton in Millville, N.J.) and gradually cooled to a cryogenic storage temperature, e.g. below −180° C., using conventional methods.

The system of the disclosure can be configured as a kit for cell banking. Such a kit generally includes a cannula, a collection element such as a syringe, a storage element such as a blood collection bag or blood collection tubes, a valve assembly, connecting tubing, and instructions for assembling the kit and using the kit to collect bone marrow during a surgical procedure and process the collected bone marrow for cell banking. The kit may also comprise filters, a transport element, and/or labels and packing material for shipping the collected bone marrow to a processing/cell banking site. The transport element typically includes a cooler type package for shipping the collected cells and can optionally include an evaporative-type cooling element. In an embodiment, the evaporative cooling element continuously evaporates small quantities of water at low pressure while the cells are in transit from the point of collection to the cell bank or other processing facility, such as a laboratory. One example of a cooler package comprising an evaporative cooling element is a NANOCOOL™ specimen shipping package (NanoPore Inc., Albuquerque, N. Mex.).

Stem cells isolated from bone marrow according the system and method of the disclosure can be used in stem cell therapies as described herein and known in the art. The stem cells are typically suspended in physiological saline solution, serum, or autologous serum for administration to a patient. The stem cells may be administered locally as an injection of a concentrated cell suspension at the site of an injury or a lesion. Alternatively, the stem cells may be administered intravenously, particularly if for some reason local administration is not practical. The number of stem cells administered to a patient for a particular treatment can vary and is dependent upon the disorder being treated, route of administration of the stem cells, and the sex, age, and health of the patient. A therapeutic dosage suitable for a microtransplant of stem cells generally includes at least 4,000,000 stem cells, preferably at least 10,000,000 cells, which can be obtained by the system and method of the disclosure without requiring, unlike conventional stem cell collection procedures, expansion of the cells to obtain a suitable number of cells.

### EXAMPLES

The following examples are illustrative and other embodiments exist and are within the scope of the invention.

### Example 1

Bone marrow was collected from three patients during hip replacement surgery. Three separate surgical approaches (anterior, posterior and lateral) were used. In each approach, bone marrow was collected from the prepared acetabulum.

In each surgery, the patient was prepared for surgery using conventional surgical methods. After dislocation of the hip and femoral neck osteotomy, the acetabulum was prepared by reaming to allow for socket ingrowth post-surgery. The reaming provided a bed of bleeding bone, from which 20 mL bone marrow (from each patient) was extracted using a syringe. The collected bone marrow was processed as described in Example 3 and the stem cells isolated from the collected bone marrow were cryogenically preserved in a cell bank as described in Example 3 for future use by the patients.

### Example 2

Bone marrow was collected from two patients during knee replacement surgery. In each surgery, bone marrow was collected from the femoral and tibial medullary canals.

In each surgery, the patient was prepared for surgery using conventional surgical methods. After osteotomy of the proximal end of the tibia and the distal end of the femur, 20 mL of bone marrow was extracted from the femoral and tibial medullary canals using a syringe. The collected bone marrow was processed as described in Example 3 and the stem cells isolated from the collected bone marrow were cryogenically preserved in a cell bank as described in Example 3 for future use by the patients.

### Example 3

Stem cells were separated and isolated from the bone marrow collected in Examples 1 and 2 and processed for cryogenic preservation and storage in a cell bank for later use by the patients. During the surgical procedures, the bone marrow was collected from each patient in Vacutainer® CPT™ tubes (available from Becton, Dickinson and Company in Franklin Lakes, N.J.) and the tubes were inverted several times to mix the collected marrow with anticoagulant contained in the tubes. The tubes were centrifuged for a minimum of 15 minutes at 1500-1800 RCF to separate the collected marrow into component layers. The stem cells are generally contained in the buffy coat layer. Therefore, the buffy layer was collected from each of the patient's collection tubes and concentrated by centrifuging the combined layers for 10 minutes at about 2,070 rpm. The supernatant was discarded and the stem cells were resuspended in 10 mL of DPBS and centrifuged again for 10 minutes at about 1,130 rpm. The supernatant was again discarded and the stem cells were resuspended in 10 mL of DPBS. An aliquot of the suspension was transferred to a Countess® Automated Cell Counter and the total number of viable cells was calculated. The suspension was centrifuged for 10 minutes at about 1,130 rpm, the supernatant discarded, and the cells were resuspended in an appropriate volume of CryoStor™ cryogenic storage medium to yield 2×10⁶-10×10⁶ cells/mL. The suspension of stem cells was transferred into cryogenic storage vials (approximately 1.0 mL per vial) and the vials were cooled at a controlled rate at approximately 1° C./min until −80° C. was obtained, before being transferred to a cryogenic freezer in cryogenic storage boxes to be stored at <−135° C. for later use by the patient.

It is important to note that the construction and arrangement of the elements of the inventions as described in this application and as shown in the figures above is illustrative only. Although some embodiments of the present inventions have been described in detail in this disclosure, those skilled in the art who review this disclosure will readily appreciate that many modifications are possible without materially departing from the novel teachings and advantages of the subject matter recited. Accordingly, all such modifications are intended to be included within the scope of the present inventions. Other substitutions, modifications, changes and omissions may be made in the design, operating conditions and arrangement of the preferred and other exemplary embodiments without departing from the spirit of the present inventions.

The system and method of the present invention can comprise conventional technology (e.g. commercially available syringes, collection bags, collection tubes, cannulas, tubing, valves, etc.) or any other applicable technology (present or future) that has the capability to perform the functions and processes/operations indicated in the FIGURES. All such technology is considered to be within the scope of the present inventions.

What is claimed is:

1. A system for collecting bone marrow for a patient during a surgical procedure, the system comprising:
a cannula operatively connected by tubing to a valve assembly;
a collection element operatively connected by tubing to the valve assembly; and
a storage element operatively connected to the valve assembly,
wherein the valve assembly comprises a selectable flow path comprising a first flow path providing fluid communication between the cannula and the collection element and a second flow path providing fluid communication between the collection element and the storage element.

2. The system of claim 1, wherein the system is a closed system.

3. The system of claim 1, wherein the collection element is a syringe.

4. The system of claim 1, wherein the storage element is a blood collection tube or a blood collection bag.

5. The system of claim 4, wherein the blood collection tube or blood collection bag comprises an anticoagulant.

6. The system of claim 1, wherein the valve assembly is manually actuated to select the first or second flow path.

7. The system of claim 1, wherein the valve assembly is an automatic valve assembly actuated by positive or negative pressure to select the first or second flow path.

8. The system of claim 7, the valve assembly comprising a first valve and a second valve wherein the second valve is closed when the first valve is opened providing a flow path between the cannula and the collection element.

9. The system of claim 8, wherein the first valve is closed when the second valve is opened providing a flow path between the collection element and the storage element.

10. The system of claim 1, further comprising a transport element.

11. The system of claim 10, wherein the transport element comprises a cooler.

12. The system of claim 11, wherein the transport element comprises an evaporative cooling element.

13. The system of claim 12, wherein the evaporative cooling element continuously evaporates small quantities of water at low pressure.

14. The system of claim 1, wherein the surgical procedure comprises a hip replacement procedure or a knee replacement procedure.

15. The system of claim 1, further comprising instructions for collecting bone marrow exposed during a surgical procedure, processing the collected bone marrow, and shipping the processed bone marrow to a cell bank.

16. The system of claim 2, wherein the closed system reduces risk of microbial contamination of bone marrow during collected during the surgical procedure.

17. A method for collecting bone marrow from a patient during a surgical procedure with the system according to claim 1, comprising:
inserting the cannula into bone marrow exposed during the surgical procedure;
actuating the valve assembly to provide a flow path between the cannula and the collection element;
suctioning the bone marrow into the collection element;
actuating the valve assembly to provide a flow path between the collection element and the storage element; and
transferring the collected marrow from the collection element to the storage element.

18. The method of claim 17, wherein the primary purpose of the surgical procedure is other than collecting bone marrow.

19. The method of claim 17, wherein the surgical procedure comprises an osteotomy.

20. The method of claim 18, wherein the surgical procedure is a hip placement procedure or a knee replacement procedure.

21. The method of claim 19, wherein the bone marrow is collected from the pelvis, femur, or tibia.

22. The method of claim 21, wherein the bone marrow is collected from the femoral canal.

23. The method of claim 21, wherein the bone marrow is collected from the tibial canal.

24. The method of claim 21, wherein the bone marrow is collected from the acetabulum or iliac crest of the pelvis.

25. The method of claim 17, further comprising centrifuging the collected bone marrow and collecting a buffy coat layer.

26. The method of claim 25, further comprising isolating stem cells from the buffy coat layer and cryogenically preserving the isolated stem cells.

27. The method of claim 26, further comprising storing the cryogenically preserved stem cells in a cell bank.
